(19) United States
(12) Patent Application Publication
(10) Pub. No.: US 2024/0065845 A1
(43) Pub. Date: Feb. 29, 2024

(54) CERVICAL ARTIFICIAL DISC AND METHOD OF CONSTRUCTING THE SAME

(71) Applicant: HealthHub Co.,Ltd., Seoul (KR)

(72) Inventors: Byung Hoon LEE, Seoul (KR); Byung Il LEE, Seoul (KR); Shi Sub BYON, Seoul (KR)

(73) Assignee: HealthHub Co.,Ltd., Seoul (KR)

(21) Appl. No.: 18/258,803

(22) PCT Filed: Dec. 22, 2020
(86) PCT No.: PCT/KR2020/018899

§ 371 Date: Jun. 21, 2023

(30) Foreign Application Priority Data
Dec. 22, 2020 (KR) 10-2020-0180762

(51) Int. Cl. A61F 2/30

(52) U.S. Cl. CPC A61F 2/30942; A61F 2002/443

(57) ABSTRACT

The present disclosure relates to a cervical artificial disc and a method of constructing the same, and to the cervical artificial disc and the method, in which an available space into which the cervical artificial disc is to be inserted is modeled by designating a plurality of landmarks, and the cervical artificial disc is designed based on the available space modeling, when designing the cervical artificial disc for a patient who requires cervical artificial disc replacement surgery.

### TECHNICAL FIELD

The present invention relates to a cervical artificial disc and a method of constructing the same, and more particularly, to the cervical artificial disc and the method, in which an available space into which the cervical artificial disc is to be inserted is modeled by designating a plurality of landmarks, and the cervical artificial disc is designed based on the available space modeling, when designing the cervical artificial disc for a patient who requires cervical artificial disc replacement surgery.

### BACKGROUND

Generally, the spine comprises the cervical vertebrae (neck bones), thoracic vertebrae (back bones), lumbar vertebrae (lower back bones), sacrum (pelvic bone), and coccyx (tailbone), and consists of a total of 33 vertebrae with seven cervical vertebrae, twelve thoracic vertebrae, five lumbar vertebrae, five sacral vertebrae, and four coccygeal vertebrae.

Between each of the cervical, thoracic, and lumbar vertebrae, there is an intervertebral disc (disc), which is a thick disc-shaped fibrous cartilage with abundant elasticity, and has a structure in which the nucleus is surrounded by a fibrous ring.

However, the disc in which the vertebrae are connected to each other at the neck, the waist, or the like can undergo degenerative changes with aging, causing decreased elasticity and reduced water content. As a result, the discs may wear out or develop cracks, leading to the loss of their primary function of shock absorption.

When the function of the discs is lost, pain is induced. If it progresses further and the fibers are severely stretched or ruptured, it can press on the nerves located behind it, causing pain in the pelvis, legs, and the like. Over time, if the space between the vertebrae becomes narrower or the vertebrae sink, spinal deformities can occur, leading to various diseases related to the spine.

The existing artificial disc was not patient-customized so that the design itself does not need to be based on the actual image of the patient. In other words, when manufactured as a ready-made product, there was no need for the design of the disc itself, and it was sufficient to check the surgical plan. However, when a patient-customized disc is to be used, a detailed disc modeling is required each time, and thus an efficient design is required. However, since a conventional cervical artificial disc has a very complicated process of manufacturing and shaping, there is a problem that the efficiency of work is reduced.

Accordingly, the present invention provides a method for modeling a space of an A-space into which the cervical artificial disc is to be inserted by using a plurality of landmarks when designing an artificial cervical disc, and easily designing a cervical artificial disc suitable for the modeled A-space.

In particular, the present invention proposes a method for modeling, in a specific joint (two unit cervical vertebrae, and a disc between them), a space of an A-space into which the cervical artificial disc is to be inserted, by setting seven landmarks in the lower end of the upper unit cervical vertebrae of the skull direction, that is, the upper cranial, 10 landmarks in the upper end of the lower unit cervical vertebrae of the spine direction, that is, the lower caudal.

Next, the prior arts existing in the technical field of the present invention are briefly described, and then the technical features that the present invention aims to achieve differentially from the prior arts are described.

First, Korean Patent No. 1137991(Apr. 12, 2012) relates to a method for manufacturing a patient-specific medical spinal implant based on an image and a spinal implant thereof. In order to solve the problem of damaging the spine during the installation of spinal implants used in spinal surgery, the method introduces the patient's image data to produce a spinal implant tailored to the individual's spinal shape, which can be inserted without damaging the spine. The implant is made of a polymer-based material and is surgically implanted using an image-based patient-customized medical spinal orthosis production method.

That is, the prior art discloses a spinal implant using biocompatible polymers to overcome the disadvantage of existing implants being metallic. By utilizing biocompatible polymers, the implant can reduce patient discomfort and has a structure that can be inserted without screw fixation into the spine, providing a minimally invasive surgical method for spinal stabilization.

The present invention, on the other hand, models the space of the A-space into which the cervical artificial disc is to be inserted by using a plurality of landmarks, and facilitates the design of a cervical artificial disc suitable for the space-modeled A-space, and thus the prior art and the present invention are clearly different from each other in terms of technical configuration.

In addition, Korean Patent No. 1936780(Jan. 3, 2019) relates to a system for designing and manufacturing a patient-customized implants for correcting spinal deformities. By applying a three-dimensional implant design and manufacturing system, optimized implants corresponding to each spinal patient's surgical site are designed and manufactured and provided to the surgeon, thereby reducing the potential for spinal deformity errors and procedural trial and error that a surgeon may encounter, and the prior art is thus related to a system and control method for designing and manufacturing patient-specific implants for correcting spinal deformities.

That is, the prior art describes a system for constructing a procedure capable of performing virtual spine deformation correction by introducing computer simulation using computational mechanics such as a finite element method in a pre-surgery planning step, and constructing a procedure capable of searching, designing, and producing an implant suitable for the spine of a patient through a simulation result, and a control method thereof.

However, the present invention models the space of the A-space into which the cervical artificial disc is to be inserted by using 7 landmarks set in the upper cranial direction and 10 landmarks set in the lower caudal direction and facilitates the design of a cervical artificial disc suitable for the space-modeled A-space. Therefore, it is clear that the present invention and the prior arts have significant structural differences.

### BRIEF SUMMARY OF THE EMBODIMENTS

The present invention is devised to solve the aforementioned problems, and it is an objective of the present invention to model a space of an A-space into which a cervical artificial disc is to be inserted by using a plurality of landmarks.

It is another objective of the present invention to design the cervical artificial disc in a height, a length, an area, a shape, or a combination thereof corresponding to the A-space modeled using a plurality of landmarks.

It is another objective of the present invention is to set seven landmarks in an upper cranial direction and set ten landmarks in a lower caudal direction so as to model a space of the A-space into which the cervical artificial disc is to be inserted.

It is another objective of the present invention is to design a patient-customized cervical artificial disc for each patient undergoing cervical artificial disc replacement surgery.

According to an embodiment of the present invention, it is characterized in that a cervical artificial disc comprises: a cranial plate configured to be coupled to an upper unit cervical vertebra; a caudal plate configured to be coupled to a lower unit cervical vertebra; and an artificial disc member configured to be coupled between the cranial plate and the caudal plate to support a longitudinal load, wherein the cranial plate, the caudal plate, and the artificial disc member are formed based on the available space in which a cervical artificial disc represented by a plurality of landmarks is implanted.

Wherein, it is characterized in that the plurality of landmarks are configured to be set a cranial anterior center, a cranial anterior right, and a cranial anterior left, at each of a center, left and right sides of a front outer periphery of a upper unit cervical vertebrae; a cranial apex at a center of the upper unit cervical vertebrae; a cranial posterior center, a cranial posterior right, and a cranial posterior left, at each of a center, left and right sides of a rear outer periphery of the upper unit cervical vertebrae; a caudal anterior center, a caudal anterior near right, a caudal anterior far right, a caudal anterior near left, and a caudal anterior far left, at each of a center, left and right sides of an anterior outer periphery of a lower unit caudal vertebrae, and a caudal posterior center, caudal posterior near right, caudal posterior far right, caudal posterior near left, and caudal posterior far left, at each of a center, left and right sides of a posterior outer periphery of the lower unit cervical vertebrae.

Wherein, it is characterized in that the available space is configured to be a space defined by the plurality of landmarks, and formed by modeling a space (A-space) between two unit cervical vertebrae to which the cervical artificial disc is implanted, and comprises a cranial edge line, which is a line connecting the cranial anterior center, cranial anterior right, cranial anterior left, cranial posterior center, cranial posterior right, and cranial posterior left; a cranial center line, which is a line connecting the cranial anterior center, apex center, and cranial posterior center, and is a center line that runs across between left and right sides of the cranial plate; an apex height, which is a distance between the cranial center line and the apex center; a caudal edge line, which is a line connecting the caudal anterior near right, caudal anterior center, caudal anterior near left, caudal posterior near right, caudal posterior center, and caudal posterior near left; a caudal center line, which is a line connecting the caudal anterior center and caudal posterior center, and a center line that runs across between left and right sides of the caudal plate; a caudal wing line, which is a line connecting the caudal anterior near right and caudal anterior far right, the caudal anterior near left and caudal anterior far left, the caudal posterior near right and caudal posterior far right, and the caudal posterior near left and caudal posterior far left; a caudal wing angle, which is an inner angle of a center vertex of a triangle with caudal wing line as a hypotenuse; and a caudal wing plate, which is a quadrilateral formed by connecting the caudal anterior near right, caudal anterior far right, caudal posterior far right, and caudal posterior near right, and the caudal anterior near left, caudal anterior far left, caudal posterior far left, and caudal posterior near left with lines.

Wherein, it is characterized in that the caudal wing angle is configured to be used for positioning the caudal plate at the center of the left and right sides of the cervical plate when implanting the caudal plate in the unit cervical vertebrae, and the caudal wing plate is used for preventing the cervical artificial disc from being rotated through friction with the unit cervical vertebra when implanting the caudal plate in the unit cervical vertebrae.

Wherein, it is characterized in that the cranial plate is configured to be manufactured to have a size capable of covering the body of the upper unit cervical vertebra, thereby preventing the cranial plate from being recessed in the unit cervical vertebra to which the cranial plate is attached, or preventing bleeding caused by the cranial plate not covering a part of the body of the unit cervical vertebra.

Moreover, it is characterized in that a method for configuring a cervical artificial disc according to an embodiment of the present invention comprises configuring a cranial plate for coupling to an upper unit cervical vertebra; configuring a caudal plate for coupling to a lower unit cervical vertebra; and configuring an artificial disc member coupled between the cranial plate and the caudal plate to support a longitudinal load, wherein the cranial plate, the caudal plate, and the artificial disc member are formed based on the available space in which a cervical artificial disc represented by a plurality of landmarks is implanted.

In addition, it is characterized in that the method further comprises setting the plurality of landmarks with a cranial anterior center, a cranial anterior right, and a cranial anterior left, at each of a center, left and right sides of a front outer periphery of a upper unit cervical vertebrae; with a cranial apex at a center of the upper unit cervical vertebrae; with a cranial posterior center, a cranial posterior right, and a cranial posterior left, at each of a center, left and tight sides of a rear outer periphery of the upper unit cervical vertebrae; with a caudal anterior center, a caudal anterior near right, a caudal anterior far right, a caudal anterior near left, and a caudal anterior far left, at each of a center, left and right sides of an anterior outer periphery of a lower unit caudal vertebrae, and with a caudal posterior center, caudal posterior near right, caudal posterior far right, caudal posterior near left, and caudal posterior far left, at each of a center, left and right sides of a posterior outer periphery of the lower unit cervical vertebrae.

Wherein, it is characterized in that the available space is a space defined by the plurality of landmarks, and formed by modeling (A-space) a space between two unit cervical vertebrae to which the cervical artificial disc is implanted, and is configured of forming a cranial edge line, which is a line connecting the cranial anterior center, cranial anterior right, cranial anterior left, cranial posterior center, cranial posterior right, and cranial posterior left; a cranial center line, which is a line connecting the cranial anterior center, apex center, and cranial posterior center, and is a center line that runs across between left and right sides of the cranial plate; an apex height, which is a distance between the cranial center line and the apex center; a caudal edge line, which is a line connecting the caudal anterior near right, caudal anterior center, caudal anterior near left, caudal posterior near right, caudal posterior center, and caudal posterior near left; a caudal center line, which is a line connecting the caudal anterior center and caudal posterior center, and a center line that runs across between left and right sides of the caudal plate; a caudal wing line, which is a line connecting the caudal anterior near right and caudal anterior far right, the caudal anterior near left and caudal anterior far left, the caudal posterior near right and caudal posterior far right, and the caudal posterior near left and caudal posterior far left; a caudal wing angle, which is an inner angle of a center vertex of a triangle with caudal wing line as a hypotenuse; and a caudal wing plate, which is a quadrilateral formed by connecting the caudal anterior near right, caudal anterior far right, caudal posterior far right, and caudal posterior near right, and the caudal anterior near left, caudal anterior far left, caudal posterior far left, and caudal posterior near left with lines.

Wherein, it is characterized in that the caudal wing angle is used for positioning the caudal plate at the center of the left and right sides of the cervical plate when implanting the caudal plate in the unit cervical vertebrae, and the caudal wing plate is used for preventing the cervical artificial disc from being rotated through friction with the unit cervical vertebra when implanting the caudal plate in the unit cervical vertebrae.

Wherein, it is characterized in that in the configuring of forming the cranial plate, the cranial plate is configured to be manufactured to have a size capable of covering the body of the upper unit cervical vertebra, thereby preventing the cranial plate from being recessed in the unit cervical vertebra to which the cranial plate is attached, or preventing bleeding caused by the cranial plate not covering a part of the body of the unit cervical vertebra.

As described above, according to the cervical artificial disc and the method for configuring the same of the present invention, a space of the A-space into which the cervical artificial disc is to be inserted is modeled by using a plurality of landmarks, and a cervical artificial disc suitable for the modeled A-space can be easily designed, so that in contrast to ready-made products, there is an effect of being able to manufacture a cervical artificial disc customized for each patient through modeling of the A-space into which the cervical artificial disc is inserted.

Furthermore, according to the present invention, a plate portion that comes into contact with the cervical vertebrae of the cervical artificial disc is allowed to be precisely designed to match the curvature of the cervical vertebrae, so that there is effects of making it easier to position the cervical artificial disc during a surgical procedure, and minimizing additional action such as bone removal to match its shape, as much as possible.

Furthermore, the present invention can be applied not only to cervical vertebrae, but also to the designs of artificial discs and artificial joints of various joint parts such as lumbar vertebrae, knees, hip joints, etc., so it has an effect of being able to be expanded and applied to other body parts.

### BRIEF DESCRIPTION OF THE DRAWINGS

FIG. 1 is a view schematically illustrating a structure of a cervical vertebrae of a human body to which the present disclosure is applied.

FIG. 2 is a detailed view illustrating a structure of an artificial cervical disc according to an embodiment of the present disclosure.

FIG. 3 is a diagram illustrating in detail an A-space represented by a plurality of landmarks used for manufacturing a cervical artificial disc according to an embodiment of the present disclosure.

FIG. 4 is a view for explaining each position of landmarks applied to the present disclosure in more detail.

FIG. 5 is a diagram illustrating in detail a structure of an A-space represented by a plurality of landmarks according to an embodiment of the present disclosure.

FIG. 6 is a view for explaining the manufacture of a cervical artificial disc using A-Space represented by a plurality of landmarks according to an embodiment of the present disclosure.

FIG. 7 is a flowchart illustrating an operational process of a method for configuring a cervical artificial disc according to an embodiment of the present invention.

FIG. 8 is a flowchart illustrating a process of generating an A-space represented by a plurality of landmarks according to an embodiment of the present invention.

### DETAILED DESCRIPTION OF THE EXEMPLARY EMBODIMENTS

Hereinafter, exemplary embodiments of the cervical artificial disc and a method for configuring the same of the present invention will be described in detail with reference to the accompanying drawings. The same reference numerals in each drawings denote the same components. In addition, specific structural or functional descriptions of embodiments of the present invention are only exemplified for the purpose of describing an embodiment according to the present invention, and unless otherwise defined, all terms used herein, including technical or scientific terms, have the same meaning as those commonly understood by one of ordinary skill in the art to which the present disclosure pertains. It should be desirable to be understood that terms, such as those defined in commonly used dictionaries, should be interpreted as having a meaning that is consistent with their meaning in the context of the relevant art, and are not to be interpreted in an idealized or overly formalized sense unless clearly defined in this specification.

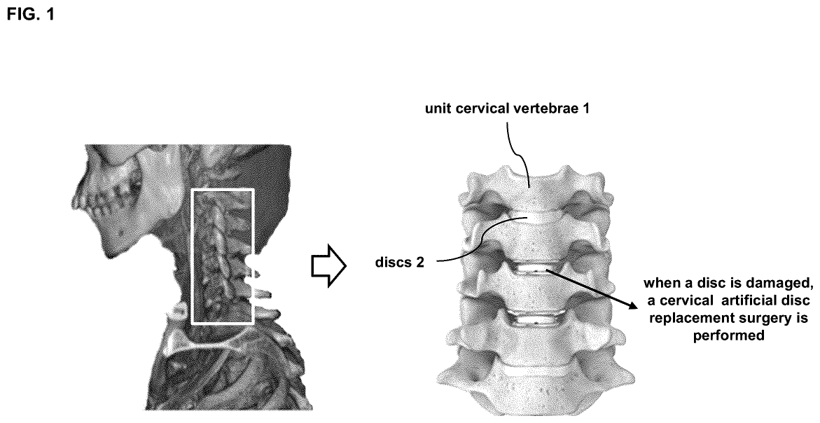

FIG. 1 is a view schematically illustrating a structure of a cervical vertebrae of a human body to which the present disclosure is applied.

As shown in FIG. 1, a cervical vertebrae of a human body comprises seven unit cervical vertebrae 1 and discs 2 are provided between the unit cervical vertebrae 1. When any of discs 2 is damaged by a wrong posture, movement, aging, disease, or the like, the corresponding disc 2 is treated with cervical artificial disc replacement surgery.

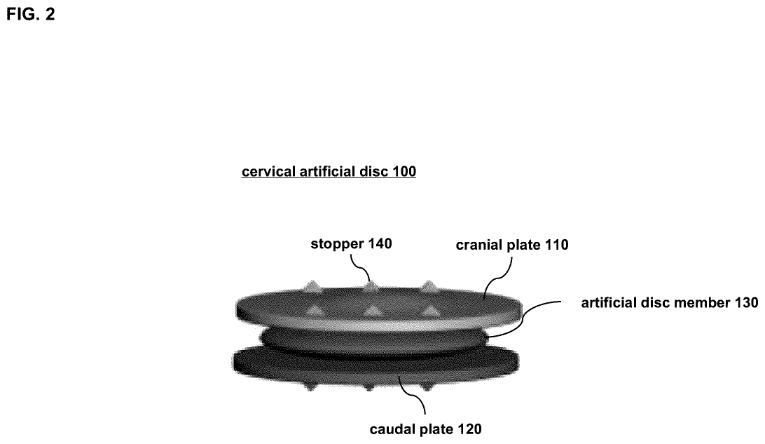

FIG. 2 is a detailed view illustrating a structure of an artificial cervical disc according to an embodiment of the present disclosure.

As shown in FIG. 2, the cervical artificial disc 100 used in the cervical artificial disc replacement comprises a cranial plate 110, a caudal plate 120, an artificial disc member 130, a stopper 140, and the like.

The cranial plate 110 is a part coupled to a unit cervical vertebra positioned in the upper direction, and at least more than one stopper 140 for firmly coupling the unit cervical vertebrae to the upper surface is provided on the upper surface thereof.

The caudal plate 120 is a part coupled to a unit cervical vertebra positioned in the lower direction, and at least more than one stopper HO for firmly coupling the unit cervical vertebrae to the lower surface is provided on the lower surface thereof.

In this case, the cranial plate 110 and the caudal plate 120 are formed to round the outer circumference and each corner, so that it is desirable to allow smoothly moved between the unit cervical vertebrae, and not to be damaged when coming into contact with the unit cervical vertebrae.

The artificial disc member 130 is coupled between the cranial plate 110 and the caudal plate 120 to perform a function of supporting a longitudinal load.

In this case, the artificial disc member 130 can be formed of various materials such as polymers, metals, and the like.

Wherein, the cranial plate 110, the caudal plate 120, and the artificial disc member 130 are formed in a shape that is most suitable for the A-space, which is an available space represented by a plurality of landmarks, based on a height (e.g., an interval between an upper vertex of the cranial plate 110 and a lower vertex of the caudal plate 120), a length (e.g., a width of a major axis), a width (e.g., a width of a minor axis), a shape (a surface or an edge), or a combination thereof.

In addition, the stopper 140 may not fall within the range of the A-space, and the cervical artificial disc may be fixed to the upper and lower cervical vertebrae.

That is, according to the present invention, when the cervical artificial disc 100 is manufactured, a plurality of landmarks are set in a medical image of a specific patient, a space into which an A-space is to be inserted is modeled based on the set landmarks, and the cervical artificial disc 100 most suitable for the space-modeled A-space with a height (e.g.,: the distance between the upper apex of the cranial plate 110 and the lower apex of the caudal plate 120), the length (e.g., the width of the major axis), the width (e.g., the width of the minor axis), the shape (a surface or an edge), or a combination thereof. Accordingly, a customized cervical artificial disc can be designed for each patient undergoing cervical artificial disc replacement surgery in accordance with the present invention.

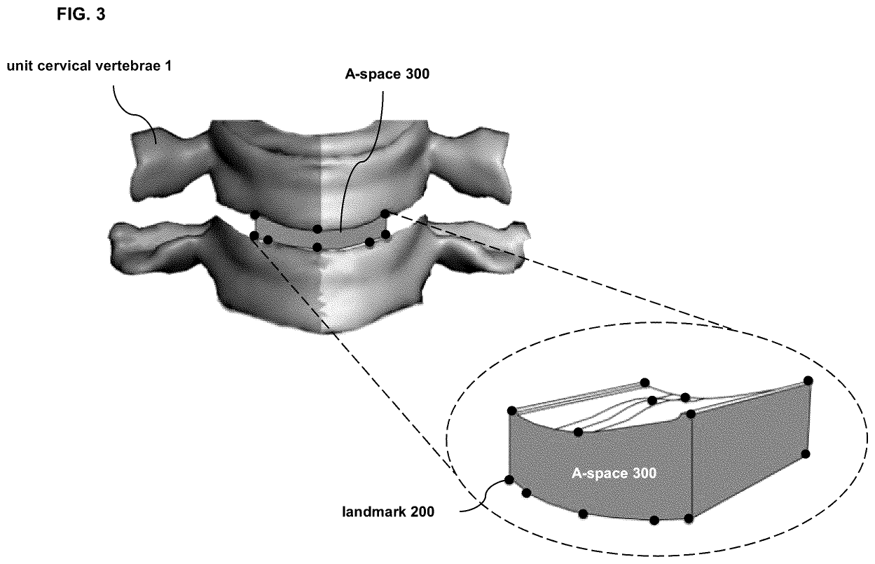

As such, configurations of the landmarks and the A-space used when manufacturing the cervical artificial disc according to the present disclosure are described in more detail with reference to FIGS. 3 to 6.

FIG. 3 is a diagram illustrating in detail an A-space represented by a plurality of landmarks used for manufacturing a cervical artificial disc according to an embodiment of the present disclosure.

As shown in FIG. 3, the A-space 300 is a space defined by a plurality of landmarks 200, and models a space between two unit cervical vertebrae 1 where the cervical artificial disc 100 is implanted. That is, when one cervical artificial disc 100 is designed, the A-space is a space defined as a limit on the size and shape of the space and is a left-right symmetric structure.

In this case, since the A-space 300 is different for each patient and the surgical site, when the cervical artificial disc 100 is manufactured by modeling the A-space 300 in the manner proposed in the present invention, it is possible to solve problems such as reduced surgical success rates, increased surgical difficulty, and decreased patient satisfaction that have been associated with the use of conventional standardized designs for cervical artificial discs.

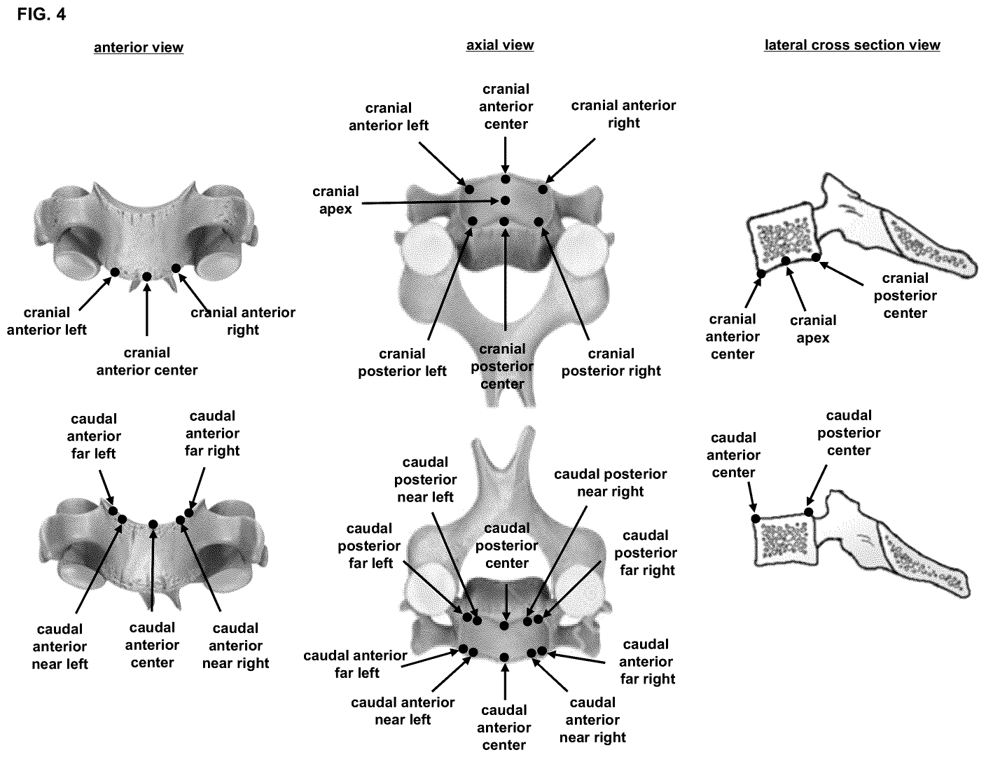

FIG. 4 is a view for explaining each position of landmarks applied to the present disclosure in more detail.

As shown in FIG. 4, the landmarks 200 are configured to secure an area to sufficiently surround the unit cervical vertebrae of the surgical site and to guide the placement of the unit cervical vertebrae of the surgical site to be implanted in alignment with the center line, and thus there are seven unit cervical vertebrae set in the cranial direction of the cervical vertebrae, and ten unit cervical vertebrae set in the caudal direction of the cervical vertebrae. In addition, the A-space 300 into which the cervical artificial disc 100 is inserted is three-dimensionally modeled by a total of 17 landmarks 200.

In other words, the landmarks 200 are configured with a cranial anterior center, a cranial anterior right, and a cranial anterior left set on center and left and right sides of the front outer periphery of the upper unit cervical vertebrae, respectively, a cranial apex act at center of the upper unit cervical vertebrae, and a cranial posterior center, a cranial posterior right, and cranial posterior left set on center and left and right sides of the rear outer periphery of the upper unit cervical vertebrae, respectively.

In addition, the landmarks 200 are configured with a caudal anterior center, a caudal anterior near right, a caudal anterior far right, a caudal anterior near left, and a caudal anterior far left, respectively, set on center and left and right of the anterior outer periphery of the lower unit caudal vertebrae, and a caudal posterior center, a caudal posterior near right, a caudal posterior far right, a caudal posterior near left and a caudal posterior far left, respectively, set on center and left and right of the rear outer periphery of the lower unit caudal vertebrae.

Meanwhile, the landmarks 200 can be directly set on a surgical site by experts (e.g., a doctor, an engineer, etc.), after identifying a medical image, such as a CT image, an MRI image, an X-ray image, and the like, or can be automatically estimated from a medical image of a user to be operated by using a pre-generated artificial intelligence learning model.

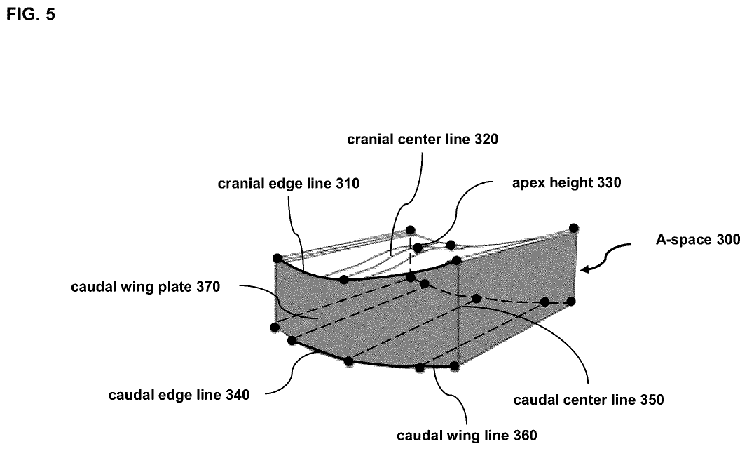

FIG. 5 is a diagram illustrating in detail a structure of an A-space represented by a plurality of landmarks according to an embodiment of the present disclosure.

As shown in FIG. 5, the A-space 300 comprises a cranial edge line 310, a cranial center line 320, an apex height 330, a caudal edge line 340, a caudal center line 350, a caudal wing line 360, a caudal wing plate 370, a caudal wing angle, and the like.

The cranial edge line 310 refers to a portion that connects he cranial anterior center, cranial anterior right, cranial anterior left, cranial posterior center, cranial posterior right, and cranial posterior left of the landmarks 200 with lines and refers to a maximum size of the cranial plate 110 of the cervical artificial disc 100.

More specifically, the cranial plate 110 of the cervical artificial disc 100 should be designed to have a size capable of coveting the body of the unit cervical vertebra as much as possible. This is because, when the cranial plate 110 is manufactured to be small, the cranial plate 110 is depressed in a corresponding unit cervical vertebra to which the cranial plate 110 is attached, and cannot normally function, or as a portion of the corresponding unit cervical vertebra body is not covered, bleeding occurs.

The cranial center line 320 is a left and right center line of the cranial plate 110, which connects a cranial anterior center, an apex center, and a cranial posterior center in the landmarks 200, with lines.

The apex height 330 is a distance between the cranial center e 320 and the apex center of the landmarks 200.

The caudal edge line 340 is a portion that connects a caudal anterior near right, a caudal anterior center, a caudal anterior near right, a caudal posterior near right, a caudal posterior center, and a caudal posterior near left of the landmarks 200 with lines.

The caudal center line 350 is a left and right center line of the caudal plate 120 in which a caudal anterior center and a caudal posterior center of the landmarks are connected to each other.

The caudal wing line 360 is a portion that connects between a caudal anterior near right and a caudal anterior far right, between a caudal anterior near left and a caudal anterior far left, between a caudal posterior near right and a caudal posterior far right, and between a caudal posterior near left and a caudal posterior far left of the landmarks 200, respectively with lines.

The caudal wing plate 370 is a portion of a rectangular form that connects among a caudal anterior near right, a caudal anterior far right, a caudal posterior far right and a caudal posterior near right, and among a caudal anterior near left, a caudal anterior far left, a caudal posterior far left and a caudal posterior near left of the landmarks 200, respectively, with lines.

The caudal wing angle is the inner angle of a center apex for a triangular form with the caudal wing line 360 as oblique.

Meanwhile, when the caudal plate 120 is implanted into the unit cervical vertebrae 1, the caudal wing angle serves to be positioned at the left and right centers of the cervical artificial disc 100.

In addition, when the caudal wing plate 370 is implanted into the unit cervical vertebra 1, the caudal wing plate 370 takes a role to prevent the cervical artificial disc 100 from being rotated through friction with the surface of the unit cervical vertebra 1.

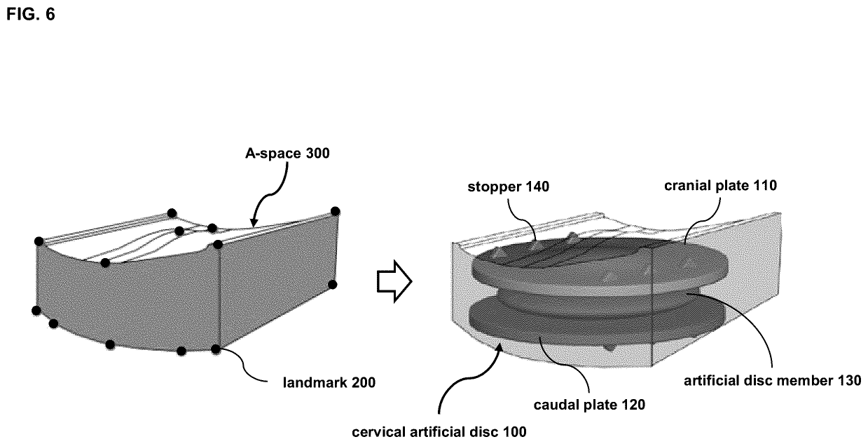

FIG. 6 is a view for explaining the manufacture of a cervical artificial disc using A-Space represented by a plurality of landmarks according to an embodiment of the present disclosure.

As shown in FIG. 6, a device in accordance with the present invention supports to set a plurality of landmarks 200 from a medical image of a specific patient for designing the cervical artificial disc, and to perform three-dimensionally modeling of the A-space 300 consisting of a plurality of landmarks 200. In addition, the cervical artificial disc 100 having the most suitable shape and size can be manufactured through the A-space 300 of the 3D modeling 300.

That is, the customized cervical artificial disc 100 can be designed for each patient, by three-dimensionally modeling the A-space 300 using the seven landmarks set in the upper cervical vertebrae and the ten landmarks set in the caudal unit cervical vertebrae.

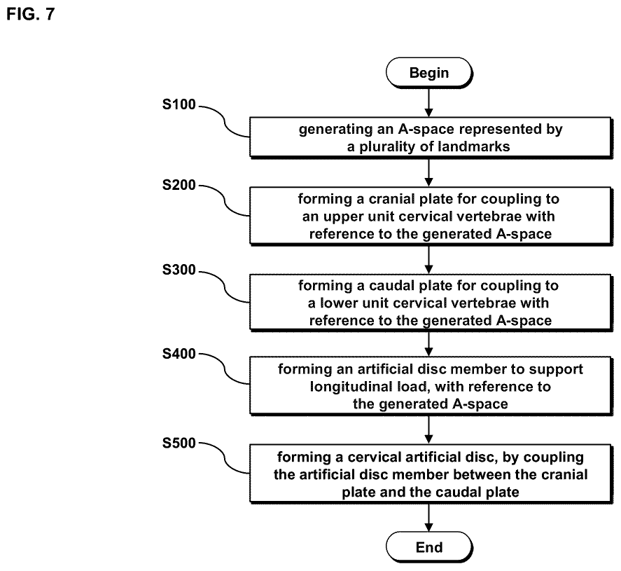

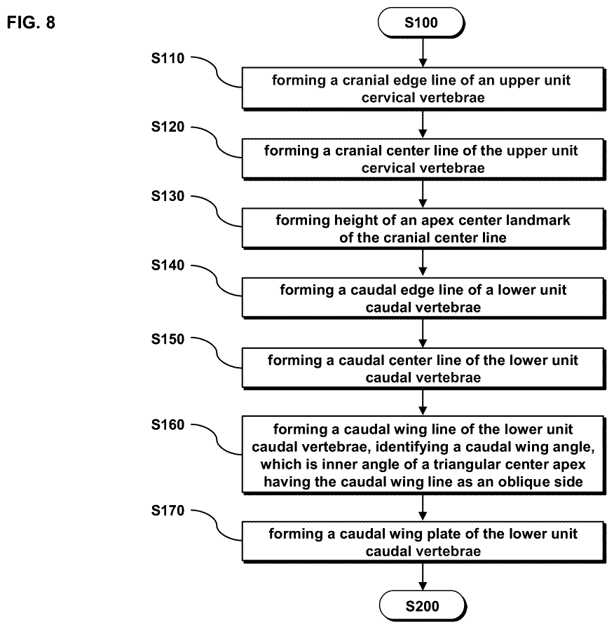

Hereinafter, an embodiment of a method for configuring a cervical artificial disc according to the present invention is described in detail with reference to FIGS. 7 and 8. In this case, each step according to the method of the present invention can be changed in order by a use environment or a person skilled in the art.

FIG. 7 is a flowchart illustrating an operational process of a method for configuring a cervical artificial disc according to an embodiment of the present invention, and FIG. 8 is a flowchart illustrating a process of generating an A-space represented by a plurality of landmarks according to an embodiment of the present disclosure.

As shown in FIG. 7, in order to configure the cervical spine artificial disc 100 according to an embodiment of the present invention, first, a plurality of landmarks 200 should be set from a medical image of a user in a device for designing a cervical artificial disc.

In this case, seven landmarks 200 are set to include a cranial anterior center, a cranial anterior right, a cranial anterior left, a cranial apex, a cranial posterior center, a cranial posterior tight, and a cranial posterior left in the upper unit cranial vertebrae, and 10 landmarks 200 are set to include a caudal anterior center, a caudal anterior near right, a caudal anterior far right, a caudal anterior near left, a caudal anterior far left, a caudal posterior center, a caudal posterior near right, a caudal posterior far right, a caudal posterior near left and a caudal posterior far left, in the lower unit caudal vertebrae.

Alter setting the plurality of landmarks 200 from the medical image of the user to be operated, the device performs generating the A-space 300 represented by the plurality of set landmarks 200, S100. That is, the A-space 300, which is a space defined by the plurality of landmarks 200, is modeled.

To provide a more detailed explanation of the S100 with reference to FIG. 8, the device first forms the cranial edge line 310 of the upper unit cervical vertebra by connecting the cranial anterior center, the cranial anterior right, the cranial anterior left, the cranial posterior center, the cranial posterior right, and the cranial posterior left, with lines, S110.

In addition, the device forms the cranial center line 320 of the upper unit cervical vertebrae, which is the left and right central lines of the cranial plate 110, by connecting the cranial anterior center, the apex center, and the cranial posterior center, with lines, S120.

Subsequently, the device forms a height between the outer edge of the cranial center line 320 and the apex center, by referring to the apex height 330 between the cranial center line 320 and the apex center, S130.

In addition, the device forms the caudal edge line 340 of the lower unit cervical vertebrae by connecting the caudal anterior near right, the caudal anterior center, the caudal anterior near right, the caudal posterior near right, the caudal posterior center, and the caudal posterior near left, with lines, S140.

In addition, the device forms the caudal center line 350 of the lower unit cervical vertebrae, which is the left and right central lines of the caudal plate 120, by connecting the caudal anterior center and the caudal posterior center, with lines, S150.

Subsequently, the device forms a caudal wing line 360 of the lower unit cervical vertebrae by connecting between the caudal anterior near right and caudal anterior far right, between the caudal posterior near right and the caudal posterior far right, and between the caudal posterior near left and the caudal posterior far left, with lines, and then identifies the caudal wing angle, which is inner angle of the triangular center apex having the caudal wing line 360 as an oblique side, S160.

In addition, the device finally completes the A-space 300 by forming a caudal wing plate 370 of the lower unit cervical vertebra with a rectangular-shape, by connecting among the caudal anterior near right, the caudal anterior far right, the caudal posterior far right and the caudal posterior near right, and among the caudal anterior near left, the caudal anterior far left, the caudal posterior far left and the caudal posterior near left, with lines, thereby finally completing the A-space 300, S170.

When the caudal plate 120 is implanted in a unit cervical vertebra, the caudal wing angle performs a function of ensuring that the caudal plate 120 can be positioned at the center of the cervical artificial disc 100 from left to right, and when the caudal plate 120 is implanted in a unit cervical vertebra, the caudal wing plate 370 performs a function of preventing the cervical artificial disc 100 from rotating through friction with the unit cervical vertebra.

Referring back to FIG. 7, after generating the A-space 300 through the S100, the device performs forming the cranial plate 110 for coupling to the upper unit cervical vertebrae with reference to the generated A-space 300, S200, forming the caudal plate 120 to be coupled to the lower unit cervical vertebrae, S300, and then sequentially forming the artificial disc member 130 coupled between the cranial plate 110 and the caudal plate 120 to support the longitudinal load, S400.

That is, the device is configured to form the cranial plate 110, the caudal plate 120, and the artificial disc member 130 with an appropriate height (e.g., the distance between the upper apex of the cladding plate 110 and the lower apex of the caudal plate 120), an appropriate length (e.g., the width of the major axis), an appropriate width (e.g., the width), an appropriate shape (surface or edge), or a combination thereof of A-space 300.

When the cranial plate 110 is formed through S200, it is needed for the device to manufacture the cranial plate 110 in a size capable of covering the body of the upper unit cervical vertebra. If the cranial plate 110 is designed to be a small size incapable of covering the body of the upper unit cervical vertebra, the cranial plate 110 is depressed in the unit cervical vertebrae to which the cranial plate 110 is attached, or bleeding occurs due to the inability to cover a portion of the corresponding unit cervical vertebrae body.

After forming the cranial plate 110, the caudal plate 120, and the artificial disc member 130 through S200 to S400, respectively, the device couples the artificial disc member 130 between the cranial plate 110 and the caudal plate 120 to finish manufacturing of the cervical artificial disc 100.

As described above, according to the present invention, a space of the A-space into which the cervical artificial disc is to be inserted is modeled by using a plurality of landmarks, and a cervical artificial disc suitable for the space-modeled A-space can be easily designed, and thus a cervical artificial disc customized for each patient unlike a ready-made product can be manufactured through modeling of the A-space into which the cervical artificial disc is inserted.

In addition, according to the present invention, since the plate portion contacting the cervical vertebrae of the cervical artificial disc can be precisely designed according to the curvature of the cervical vertebrae, it is possible to facilitate positioning of the cervical artificial disc during a surgical procedure, and to maximally suppress additional measures such as bone reshaping to fit the shape.

In addition, the present invention can be applied to artificial disks and artificial joint designs of various joint parts such as lumbar vertebrae, knees, hip joints, etc. as well as cervical vertebrae, and thus can be expanded and applied to other body parts.

As described above, the present invention has been described with reference to the embodiment shown in the drawings, but this is merely an example, and it will be understood by a person skilled in the art that various modifications and other equivalent embodiments are possible therefrom. Therefore, the technical scope of the present invention should be determined by the following claims.

The present invention models a space of an A-space into which a cervical artificial disc is to be inserted by using a plurality of landmarks, and easily designs a cervical artificial disc suitable for the space-modeled A-space, thereby manufacturing a cervical artificial disc customized for each patient, unlike a ready-made product through modeling of the A-space into which the cervical artificial disc is inserted.

What is claimed is:

1. A cervical artificial disc comprising:
a cranial plate configured to be coupled to an upper unit cervical vertebra;
a caudal plate configured to be coupled to a lower unit cervical vertebra; and
an artificial disc member configured to be coupled between the cranial plate and the caudal plate to support a longitudinal load,
wherein the cranial plate, the caudal plate, and the artificial disc member are formed based on an available space in which the cervical artificial disc represented by a plurality of landmarks is implanted.

2. The cervical artificial disc of claim 1, wherein the plurality of landmarks are configured to be set:
a cranial anterior center, a cranial anterior right, and a cranial anterior left, in a center, left and right sides of a front outer region of the upper unit cervical vertebrae;
a cranial apex in a center of the upper unit cervical vertebrae;
a cranial posterior center, a cranial posterior right, and a cranial posterior left, in a center, left and right sides of a rear outer region of the upper unit cervical vertebrae;
a caudal anterior center, a caudal anterior near right, a caudal anterior far right, a caudal anterior near left, and a caudal anterior far left, in a center, left and right sides of an anterior outer region of a lower unit caudal vertebrae; and
a caudal posterior center, caudal posterior near right, caudal posterior far right, caudal posterior near left, and caudal posterior far left, at each of a center, left and right sides of a posterior outer periphery of the lower unit cervical vertebrae.

3. The cervical artificial disc of claim 2, wherein the available space is a space defined by the plurality of landmarks, and models an A-space between two unit cervical vertebrae to which the cervical artificial disc is implanted, and comprises:
a cranial edge line, which is a line connecting the cranial anterior center, cranial anterior right, cranial anterior left, cranial posterior center, cranial posterior right, and cranial posterior left;
a cranial center line, which is a line connecting the cranial anterior center, apex center, and cranial posterior center, and is a center line that runs across between left and right sides of the cranial plate;
an apex height, which is a distance between the cranial center line and the apex center;
a caudal edge line, which is a line connecting the caudal anterior near right, caudal anterior center, caudal anterior near left, caudal posterior near right, caudal posterior center, and caudal posterior near left;
a caudal center line, which is a line connecting the caudal anterior center and caudal posterior center, and a center line that runs across between left and right sides of the caudal plate;
a caudal wing line, which is a line connecting the caudal anterior near right and caudal anterior far right, the caudal anterior near left and caudal anterior far left, the caudal posterior near right and caudal posterior far right, and the caudal posterior near left and caudal posterior far left;
a caudal wing angle, which is an inner angle of a center vertex of a triangle with caudal wing line as a hypotenuse; and
a caudal wing plate, which is a quadrilateral formed by connecting the caudal anterior near right, caudal anterior far right, caudal posterior far right, and caudal posterior near right, and the caudal anterior near left, caudal anterior far left, caudal posterior far left; and caudal posterior near left with lines.

4. The cervical artificial disc of claim 3, wherein the caudal wing angle is used for positioning the caudal plate at the center of the left and right sides of the cervical plate when implanting the caudal plate in the unit cervical vertebrae; and
wherein the caudal wing plate is used for preventing the cervical artificial disc from being rotated through friction with the unit cervical vertebra when implanting the caudal plate in the unit cervical vertebrae.

5. The cervical artificial disc of claim 1, wherein the cranial plate is configured to be manufactured to have a size capable of covering the body of the upper unit cervical vertebra, thereby preventing the cranial plate from being recessed in the unit cervical vertebra to which the cranial plate is attached, or preventing bleeding caused by the cranial plate not covering a part of the body of the unit cervical vertebra.

6. A method for constructing a cervical artificial disc, the method comprising:
configuring a cranial plate for coupling to an upper unit cervical vertebrae;
configuring a caudal plate for coupling to a lower unit cervical vertebrae; and
configuring an artificial disc member coupled between the cranial plate and the caudal plate to support a longitudinal load,
wherein the cranial plate, the caudal plate, and the artificial disc member are formed based on the available space in which a cervical artificial disc represented by a plurality of landmarks is implanted.

7. The method of claim 6, wherein the method further comprises:
setting the plurality of landmarks of a cranial anterior center, a cranial anterior right, and a cranial anterior left, at each of a center, left and right sides of a front outer periphery of a upper unit cervical vertebrae, a cranial apex at a center of the upper unit cervical vertebrae, a cranial posterior center, a cranial posterior right, and a cranial posterior left, at each of a center, left and right sides of a rear outer periphery of the upper unit cervical vertebrae; and
setting the plurality of landmarks of a caudal anterior center, a caudal anterior near right, a caudal anterior far right, a caudal anterior near left, and a caudal anterior far left, at each of a center, left and right sides of an anterior outer periphery of a lower unit caudal vertebrae, and a caudal posterior center, caudal posterior near right, caudal posterior far right, caudal posterior near left, and caudal posterior far left, at each of a center, left and right sides of a posterior outer periphery of the lower unit cervical vertebrae.

8. The method of claim 7, wherein the available space is a space defined by the plurality of landmarks, and models an A-space between two unit cervical vertebrae to which the cervical artificial disc is implanted, and is configured to form:
a cranial edge line, which is a line connecting the cranial anterior center, cranial anterior right, cranial anterior left, cranial posterior center, cranial posterior right, and cranial posterior left;
a cranial center line, which is a line connecting the cranial anterior center, apex center, and cranial posterior center, and is a center line that runs across between left and right sides of the cranial plate;
an apex height, which is a distance between the cranial center line and the apex center;
a caudal edge line, which is a line connecting the caudal anterior near right, caudal anterior center, caudal anterior near left, caudal posterior near right, caudal posterior center, and caudal posterior near left;
a caudal center line, which is a line connecting the caudal anterior center and caudal posterior center, and a center line that runs across between left and right sides of the caudal plate;
a caudal wing line, which is a line connecting the caudal anterior near right and caudal anterior far right, the caudal anterior near left and caudal anterior far left, the caudal posterior near right and caudal posterior far right, and the caudal posterior near left and caudal posterior far left;
a caudal wing angle, which is an inner angle of a center vertex of a triangle with caudal wing line as a hypotenuse; and
a caudal wing plate, which is a quadrilateral formed by connecting the caudal anterior near right, caudal anterior far right, caudal posterior far right, and caudal posterior near right, and the caudal anterior near left, caudal anterior far left, caudal posterior far left, and caudal posterior near left with lines.

9. The method of claim 8, wherein the caudal wing angle is used for positioning the caudal plate at the center of the left and right sides of the cervical plate when implanting the caudal plate in the unit cervical vertebrae; and
wherein the caudal wing plate is used for preventing the cervical artificial disc from being rotated through friction with the unit cervical vertebra when implanting the caudal plate in the unit cervical vertebrae.

10. The method of claim 6, wherein, in configuring the cranial plate, the cranial plate is configured to be manufactured to have a size capable of covering the body of the upper unit cervical vertebra, thereby preventing the cranial plate from being recessed in the unit cervical vertebra to which the cranial plate is attached, or preventing bleeding caused by the cranial plate not covering a part of the body of the unit cervical vertebra.
